(19) United States
(12) Patent Application Publication
(10) Pub. No.: US 2008/0234629 A1
(43) Pub. Date: Sep. 25, 2008

(54) WATER ELECTROLYSIS TO FACILITATE DRUG DELIVERY BY IONTOPHORESIS

(76) Inventor: John Petersen, Acton, MA (US)

Correspondence Address: ELMORE PATENT LAW GROUP, PC, 515 Groton Road, Unit 1R, Westford, MA, 01886, US

(21) Appl. No.: 12/052,952

(22) Filed: Mar. 21, 2008

Related U.S. Application Data
(60) Provisional application No. 60/919,356, filed on Mar. 22, 2007.

(51) Int. Cl. A61N 1/30

(52) U.S. Cl. 604/20

(57) ABSTRACT

The present invention is an iontophoretic system that electrolyzes water to enhance the iontophoretic delivery of an active agent. The invention provides a cartridge adapted for use in iontophoretic system comprising electronics associated with a working electrode capable of electrolyzing water and a conductive composition comprising water and at least one active agent wherein the conductive composition has an initial pH in the absence of the flow of current, wherein upon flow of current through the electrode, water is electrolyzed, and the pH of the composition is changed to a second pH thereby providing enhanced iontophoretic delivery of the active agent.

### RELATED APPLICATION

This application claims the benefit of U.S. Provisional Application No. 60/919,356, filed on Mar. 22, 2007. The entire teachings of the above application are incorporated herein by reference.

### BACKGROUND OF THE INVENTION

Iontophoresis has been employed for many years as a means for applying medication locally through a patient's skin and for delivering medicaments to the eyes and ears. The application of an electric field to the skin has been shown known to enhance the skin's permeability to various pharmaceutical agents. The use of iontophoretic transdermal delivery techniques obviates the need for hypodermic injection for many medicaments, thereby eliminating the concomitant problems of trauma, pain and risk of infection to the patient.

Iontophoresis involves the application of an electromotive force to drive or repel oppositely charged ions through the dermal layers into a target tissue or treatment site. Particularly suitable target tissue include tissues adjacent to the delivery site for localized treatment, and tissues remote therefrom in which case the medicament enters into the circulatory system and is transported to a tissue by the blood. Positively charged ions are driven into the skin at an anode while negatively charged ions are driven into the skin at a cathode. Studies have shown increased skin penetration of drugs at anodic or cathodic electrodes regardless of the predominant molecular ionic charge on the drug. This effect is mediated by polarization and osmotic effects.

Regardless of the charge of the medicament to be administered, an iontophoretic delivery device employs two electrodes (an anode and a cathode) in conjunction with the patient's skin to form a closed circuit between one of the electrodes (referred to herein alternatively as a “working” or “application” or “applicator” electrode) which is positioned at the delivered site of drug delivery and a passive or “grounding” electrode affixed to a second site on the skin to enhance the rate of penetration of the medicament into the skin adjacent to the applicator electrode.

Various approaches have been taken to increase the drug delivery efficiency (i.e. the amount of drug delivered per unit of applied electrical current) of transdermal drug or active agent delivery. Transdermal drug delivery can be improved, for example, by increasing the driving force from the drug formulation into the skin. Higher driving forces result in shorter treatment times increasing patient convenience and compliance. The electric gradient employed in traditional iontophoresis provides the driving force to the charged drug formulation to move the drug into tissue. In general, increasing the amount of current produces a corresponding increase in iontophoretic transport up to point, after which the response plateaus and further increase in the current has no effect. Typically, the driving force for drug delivery can also be increased by increasing drug concentration. However, the physiochemical properties of the drug to be delivered will affect its particular response to increased current and concentration.

The importance of pH has been discussed in the literature (see Banga, A. K., et. al., Journal of Controlled Release, 1988, 7, p. 1-24 and Yogeshvar N. K., et al., Advanced Drug Delivery Reviews, 2004, 56, p. 619-658). However, generally, most iontophoretic devices are designed to avoid pH shifts by using Ag/AgCl electrodes which prevent the electrolysis of water.

There continues to be a need for enhanced iontophoretic drug delivery methods.

### SUMMARY OF THE INVENTION

The invention provides a product, such as a cartridge adapted for use in an iontophoretic device, comprising a conductive, aqueous formulation comprising at least one active agent and one or more electrodes capable of hydrolyzing water which alters the pH of the formulation during drug delivery resulting in enhanced drug delivery and/or improved formulation stability during storage. The invention is suitable for incorporation into iontophoretic drug products and usable with iontophoretic devices.

### DETAILED DESCRIPTION OF THE INVENTION

The present invention provides a device adapted for use in an iontophoretic system comprising a working electrode capable of electrolyzing water and a conductive composition containing water and one or more active agents wherein the composition has an initial pH in the absence of the flow of current, wherein upon flow of current through the electrode, water is electrolyzed, and the pH of the composition is changed to a second pH. Thus, the water is present in an amount effective to change the initial pH of the composition upon flow of current through the electrode to a second pH. As used herein the term “conductive composition” means any pharmaceutical composition comprising water and at least one active agent and is capable of carrying a current useful for iontophoresis (for example, from about 0.1 mA to about 10 mA) at an applied voltage of less than about 100V. The pH of a composition is “changed to a second pH” if the pH is shifted either up or down by at least about 1, preferably at least about 2, even more preferably by at least about 3 or more pH units.

In most iontophoresis devices currently in use, at least two electrodes are used. Both of these electrodes are disposed so as to be in intimate electrical contact with some portion of the skin of the body. One electrode, called the active or donor electrode is the electrode from which the active agent is delivered into the body via the skin by iontophoresis. The other electrode, called the counter or return electrode, serves to close the electrical circuit through the body. In conjunction with the patient's skin contacted by the electrodes, the circuit is completed by connection of the electrodes to a source of electrical energy, e.g., a battery; and usually to circuitry capable of controlling current passing through the device. For example, if the ionic substance to be driven into the body is positively charged, then the positive electrode (the anode) will be the active electrode and the negative electrode (the cathode) will serve to complete the circuit. If the ionic substance to be delivered is negatively charged, then the cathodic electrode will be the active electrode and the anodic electrode will be the counter electrode.

Furthermore, iontophoretic devices currently in use generally require a reservoir or source of the active agent, preferably an ionized or ionizable species (or a precursor of such species) which is to be iontophoretically delivered or introduced into the body. Such drug reservoirs are connected to the anode or the cathode of an iontophoresis device to provide a fixed or renewable source of one or more desired species or active agents. Preferred iontophoretic delivery devices useful with the compositions and methods of the invention include but are not limited to those described in U.S. Pat. Nos. 6,148,231, 6,385,487, 6,477,410, 6,553,253, and U.S. Patent Publication Numbers 2004/0111051, 2003/0199808, 2004/0039328, 2002/0161324, the contents of which are herein incorporated by reference.

The reservoir or similar structure that contains the active agent to be delivered can be in the form of any material suitable for making contact between the iontophoresis unit and the skin. Suitable materials include, but are not limited to, foams, ion exchange resins, gels and matrices. Iontophoresis gels include, but are not limited to, karaya gum, other polysaccharide gels, or similar hydrophilic aqueous gels capable of carrying ions. Specific examples of such gels include polyethylene oxide, polyvinyl alcohol, polyvinyl pyrrolidone, methyl cellulose, hydroxyethylcellulose, polyhydroxyethyl methacrylate, polyhydroxyethyl methacrylate; alginates, agarose, polyacrylamide, and the like.

An “active agent” refers to the entity to be delivered by iontophoresis. The active agent can be any chemical (for example, a small molecule) or a biological (for example, a protein or antibody) that may be used on, or administered to a humans or other animal as an aid in the diagnosis, treatment or prevention of disease or other abnormal or cosmetic condition or for the relief of pain or to control or diagnose or alleviate or improve any physiologic or pathologic condition. In another embodiment, the active agent is a pharmacologically active agent. As used herein, the term “active agent” may be used interchangeably with the terms, “drug”, “pharmaceutical”, “medicament,” “drug substance,” or “therapeutic”. As used herein the “active agent” encompasses natural or homeopathic products that are generally not considered therapeutic, such as inks and pigments for tattoos. It is to be understood that an “active agent” can be any substance capable of electrokinetic transport into a body surface, such as the skin or mucocutaneous membrane, e.g. into or from a treatment site for diagnostic or therapeutic purposes.

Active agents of the invention include, but are not limited to, biologically active compounds or a mixture of compounds that have a therapeutic, prophylactic pharmacological and/or physiological effect in a patient or recipient. In one embodiment, the active agent is sufficiently potent such that it can be delivered into a body surface such as the skin or other membrane of the patient in a sufficient quantity to produce a desired result (for example, treatment or alleviation of a condition). The active agent for use in the method of the invention can be delivered alone, or as a prodrug, or in combination with other therapeutics or substances. Other substances can include pharmaceutically acceptable carriers or excipients, permeation enhancers, water, hydrogels, buffers, bacteriostatics, stabilizers, antioxidants, colorants, opaques, other active agents and the like. As used herein, the terms “penetration enhancer” and “permeation enhancer” are used interchangeably herein. A “permeation enhancer” is a material which achieves permeation enhancement or an increase in the permeability of the body surface to the active agent. As used herein, pharmaceutically acceptable carriers and excipients include any non-toxic diluent or other formulation auxiliary that is suitable for use in iontophoresis. Examples of pharmaceutically acceptable carriers or excipients include but are not limited to a solvent, cosolvents, solubilizing agents, buffers, pharmaceutically acceptable bases and alcohols.

In a preferred embodiment, the iontophoretic system comprises at least one electrode that causes the electrolysis of water at the anode or the cathode. At the anode, protons will be generated resulting in protonation of any basic substances present in the system and reducing the pH of the system solution as shown in the equation:

2H₂O→O₂+4H⁺+4e⁻

At the cathode, hydroxide ions will be generated resulting in the deprotonation of any acids present and increasing the pH of the solution as shown in the equation:

4H₂O+4e⁻→O₂+2H₂+4OH⁻

Suitable electrode materials for the working electrode are stainless steel, platinum, nickel, gold and carbon. The counter electrode may be made from platinum, nickel, gold, carbon, silver, silver chloride or other materials suitable for electrodes known to the art.

In another embodiment, the invention provides a device adapted for use in an iontophoretic system wherein the conductive composition comprises water in an amount at least about 10% by weight, preferably at least about 30 to about 60% by weight. In another embodiment, the conductive composition is a cream and the composition comprises water in an amount of at least about 50% by weight of the cream, preferably at least about 60 to about 90% by weight of the cream.

In yet another embodiment, the invention provides a device adapted for use in iontophoretic system or device further comprising a surfactant, wetting agent or penetration enhancer. In one embodiment, the surfactant is an ionic surfactant having a charge that is the same as that of the active agent to be delivered. In another embodiment, the surfactant is present in the device in an amount less than about 0.2% by weight, more preferably less than about 0.1%. Suitable ionic surfactants include, but are not limited to, sodium dodecylsulfate (SDS), an anionic surfactant, centrimide, and/or a cationic surfactant.

In yet another embodiment, the conductive composition of the invention is in the form of a solution or a cream.

In one embodiment, the conductive composition comprises an active agent, water and a viscosity modulating agent wherein the initial pH prior to iontophoretic delivery of the composition is about 7 (neutral), and the second pH after onset of the flow of current during electrophoretic delivery is an alkaline pH. In one embodiment, the second pH is about 9. In yet another embodiment, the second pH is about 10. In an additional embodiment, the conductive composition comprises an active agent, water and viscosity modulating agent wherein the initial pH is about 7 and the second pH is an acidic pH. In one embodiment, the second pH is less than about 4. In another embodiment, the second pH is between about 2.5 and 3.5.

A viscosity modifying agent includes any agent that is capable of modulating the viscosity of the conductive composition. Viscosity modifying agents useful in the practice of the invention include, but are not limited to, ionic and non-ionic, high viscosity, water soluble polymers; crosslinked acrylic acid polymers such as the “carbomer” family of polymers, e.g., carboxypolyalkylenes that may be obtained commercially under the Carbopol® trademark; hydrophilic polymers such as polyethylene oxides, polyoxyethylene-polyoxypropylene copolymers, and polyvinylalcohol; cellulosic polymers and cellulosic polymer derivatives such as hydroxypropyl cellulose, hydroxyethyl cellulose (HEC), hydroxypropyl methylcellulose, hydroxypropyl methylcellulose phthalate, methyl cellulose, carboxymethyl cellulose, and etherified cellulose; gums such as tragacanth and xanthan gum; sodium alginate, calcium alginate; gelatin, hyaluronic acid and salts thereof, chitosans, gellans or any combination thereof.

In another embodiment, the conductive composition comprises an immobile matrix, such as a porous pad, permeated with a solution comprising the active agent, water and a viscosity modulating agent wherein the initial pH of the composition is preferably about 7 (neutral), and the second pH is either alkaline or acidic. In one embodiment, the second pH is at least about 9, or at least about 10. In another embodiment, the second pH is less than about 5 or less than about 4, or between about 2.5 and about 3.5.

In a further embodiment, the active agent is acyclovir.

Pre-packaged drug substances generally must be stable for months under routine storage conditions, but need only be stable under the iontophoretic delivery conditions for minutes or hours. In one embodiment, the active agent, excipient, or surfactant used in accordance with the invention may be pH unstable at the second pH which results from iontophoretic delivery using the device of the invention. For example, in the case of a drug substance whose stability varies as a function of pH, a drug formulation comprising the pH unstable drug substance could be manufactured and/or stored at a pH at which the drug is stable, thereby preserving the stability of pharmaceutical agent in the formulation during storage and handling. The device of the invention is particularly useful in delivering pH unstable pharmaceutical agents since the drug only needs to be stable at the second pH during the iontophoretic treatment. Use of a device of the invention effectively bypasses any issues resulting from the pH instability of the pharmaceutical agent. As used herein, an active agent, excipient, or surfactant is pH unstable when the active agent, excipient, or surfactant has decreased activity or potency at one pH compared to that at another pH. For example, an active agent is pH unstable when the active agent shows decreased activity or potency at acidic pH when compared to neutral pH. As will be appreciated by one of skill in the art, decreased activity or potency can be the result of chemical or physical degradation of the active agent, excipient or surfactant. An active agent, excipient, or surfactant is stable at a pH at which there is no or substantially no decrease in activity or potency due to pH. In one embodiment, the active agent is pH unstable.

Likewise, the active agent, or other component of the conductive formulation used in accordance with the invention may have other characteristics at the second pH resulting from electrolysis of water during iontophoretic delivery using the device of the invention. Such characteristics may be undesirable during preparation and storage and handling of the active agent or other components of the conductive composition, but would be acceptable during iontophoresis with the device of the invention due to the short period of time necessary to deliver the active agent or other component exhibiting such characteristics. For example, an active agent can become corrosive at the second pH when current is applied while using the device in iontophoretic delivery. However, during preparation, storage and handling, such corrosive active agent may conveniently be maintained at a pH at which the active agent is not corrosive until such time that it is iontophoretically delivered in accordance with the device of the invention.

In another embodiment, the invention provides a cartridge adapted for use in an iontophoretic system comprising an electrode capable of electrolyzing water and a conductive composition comprising at least one active agent and water, wherein the device is capable of altering the pH in the iontophoretic system from its initial pH to a second pH at which the ionic strength of the active agent or composition is increased to enhance iontophoretic delivery. In one embodiment, the active agent or composition is not substantially ionized at the initial pH and is ionized at the second pH.

In another preferred embodiment, the invention provides a device adapted for use in an iontophoretic system comprising an electrode capable of electrolyzing water and a conductive composition containing one or more active agents and water, wherein the device is capable of altering the pH to a pH suitable for initiation of a catalytic reaction. This feature is particularly desirable when, for example, a pH sensitive hydrogel is used to package the conductive composition of the invention. Acid or base created by electrolysis during the use of the device of the invention in iontophoresis could be used to create a pH change that would depolymerize the hydrogel and reduce the formulation viscosity at the time of delivery.

In another embodiment, the invention provides a device adapted for use in an iontophoretic system comprising an electrode capable of electrolyzing water and a conductive composition containing one or more active agents and water, wherein the device is capable of altering the pH to a second pH suitable for initiation of a chemical reaction.

In yet another embodiment, the invention provides a device adapted for use in an iontophoretic system comprising an electrode capable of electrolyzing water and a conductive composition containing one or more prodrugs of active agents and water, wherein the device is capable of altering the pH to a pH suitable for deprotection of the prodrug.

In another preferred embodiment, the invention provides a device adapted for use in iontophoretic system comprising an electrode capable of electrolyzing water and a conductive composition containing one or more active agents wherein the active agent is encapsulated in a acid or base labile polymer coating, wherein the device is capable of altering the pH to an effective level to initiate depolymerization of the acid or base labile polymer coating.

The advantages of the above approaches are numerous and include, for example ease of manufacture, particularly with regard to unstable active agents, and improved stability during storage, particularly of unstable active agents.

In another embodiment, the invention provides a device adapted for use in an iontophoretic system comprising an electrode capable of electrolyzing water and a conductive composition containing one or more active agents wherein the device functions to maintain the pH during iontophoresis. For example, in anodal iontophoresis in which the acid is being transported out of the drug delivery reservoir, water electrolysis can be used to replace the acid being lost and prevent a gradual rise in pH during drug delivery. Cathodal delivery would behave in the opposite sense.

The invention further comprises an iontophoretic device comprising a housing, an anode, a cathode operably linked to the anode and an iontophoretic pharmaceutical composition according to the invention in contact with either the anode or the cathode. Non-limiting examples of preferred iontophoretic devices are disclosed in U.S. Pat. No. 6,477,410.

The invention additionally comprises methods of delivering an active agent comprising administering an active agent with an iontophoretic device comprising a housing, an anode, a cathode operably linked to the anode and an iontophoretic pharmaceutical composition according to the invention in contact with either the anode or the cathode.

### EXAMPLES

### Example 1

### Iontophoretic System Based on Acyclovir Cream (Table 1)

Acyclovir cream is not buffered; the measured pH of the cream is about 6 to 7. At this pH acyclovir is a neutral molecule. Acyclovir accepts a proton under acidic conditions to become positively charged (pKa of the conjugate acid is 2.27) and loses a proton under basic conditions (pKa 9.25). The only ionized species in the acyclovir cream formulation is sodium dodecyl sulfate.

TABLE 1
Acyclovir 5% Cream Composition

| | | Composition |
|---|---|---|
| Ingredients | Function | (% w/w) |
| Active Ingredient | | |
| Acyclovir | Active | 5.00 |
| Other Ingredients | | |
| Propylene Glycol | Solvent | 40.00 |
| White Petrolatum | Emollient | 12.50 |
| Cetostearyl Alcohol | Emulsifying Agent | 6.75 |
| Light Mineral Oil | Emollient | 5.00 |
| Polaxamer 407 | Wetting Agent/Stabiliser | 1.00 |
| Sodium Dodecyl Sulfate | Emulsifying Agent | 0.75 |
| Water, Purified | Vehicle | TO 100.00 |

The device contains the cream in a pad backed by a stainless steel electrode. The electrode in contact with the cream in this application is the anode. During the use of the device, a current of 0.4 mA is passed through the cream for 10 minutes. Because of the use of a stainless steel electrode, electrolysis of water occurs, generating protons at the anode. Since acyclovir is the only base present in the system, acyclovir ions are formed. These ions are transported by the electric field while the neutral acyclovir can only be transported by electroosmosis.

The pH of the drug cartridge after iontophoretic treatment (0.4 mA for 600 seconds) ranges from 2.5 to 3.5 (in vivo experiments with rabbits, in vitro experiments with Franz cells). This confirms that acid is being generated in the drug cartridge and is contributing to the formation of acyclovir cations. The cations are actively transported by the electric field contributing to the iontophoretic drug delivery process.

The generation of protons through the electrolysis of water is significant relative to the amount of acyclovir in the formulation. The solubility of acyclovir is the aqueous portion of the formulation is estimated to be 0.3% by weight from propylene glycol/water solubility measurements. The soluble acyclovir in a cartridge filled with approximately 190 mg of cream is 1.6×10⁶ moles (190 mg×70% aqueous phase×0.0027 solubility÷225 g/mole (MW of acyclovir)). Assuming that the electrolysis is 100% efficient, the expected proton generation is 2.5×10⁶ moles (0.4 mA×600 sec×6.2×10¹⁸, coulomb, ÷6.2×10²³ mole; assuming each electron generate one proton). Thus the electrolysis of water provides enough protons to ionize the soluble portion of the dose. Since the current density in the invented system is typical for iontophoresis, the rate of proton generation in this system can be realized in other iontophoretic systems. Because the coulombic dose in this iontophoretic system is at the low end of iontophoretic doses, larger quantities of protons could be generated for use in other applications.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

What is claimed is:

1. A cartridge adapted for use in an iontophoretic system comprising a working electrode capable of electrolyzing water and a conductive composition comprising water and at least one active agent and an optional excipient, wherein the conductive composition has an initial pH and wherein the water is present in an amount effective to change the initial pH of the composition upon flow of current through the electrode to a second pH.

2. The cartridge of claim 1 wherein the composition is stable at the initial pH.

3. The cartridge of claim 1 wherein the active agent and/or the excipient is not substantially ionized at the initial pH and is ionized at the second pH.

4. The cartridge of claim 1 wherein the solubility of the active agent at the second pH is greater than that at the initial pH.

5. The cartridge of claim 1 wherein the composition is corrosive at the second pH.

6. The cartridge of claim 1 wherein the composition is unstable at the second pH.

7. The cartridge of claim 1 wherein the active agent is unstable at the second pH.

8. The cartridge of claim 1 wherein the formulation further comprises an excipient which is unstable at the second pH.

9. The cartridge of claim 1 wherein the second pH is alkaline.

10. The cartridge of claim 9 wherein the initial pH is between about 2 and about 10.

11. The cartridge of claim 1 wherein the active agent undergoes a chemical reaction at the second pH.

12. The cartridge of claim 1 wherein the active agent is a prodrug and undergoes deprotection of the prodrug at the second pH.

13. The cartridge of claim 1 wherein when the electrode is a cathode wherein the cathode is made of a material selected from the group consisting of stainless steel, platinum and nickel.

14. The cartridge of claim 1 wherein the electrode is an anode and wherein the anode is made of a material selected from the group consisting of stainless steel, platinum, nickel, or carbon.

15. The cartridge of claim 13 wherein the water is present in an amount of at least about 10% by weight.

16. The cartridge of claim 15 wherein the water is present in an amount of at least about 30%.

17. The cartridge of claim 14 wherein the water is present in an amount of at least about 10% by weight.

18. The cartridge of claim 17 wherein the water is present in an amount of at least about 30%.

19. The cartridge of claim 1 wherein the conductive composition is a solution or cream.

20. The cartridge of claim 1 further comprising an immobile matrix permeated with the active agent.

21. An iontophoretic device comprising a housing, the cartridge according to claim 1 and a grounding electrode.

22. A method for delivering an active agent to a patient comprising administering an active agent with an iontophoretic device according to claim 21.
